(19) United States
(12) Patent Application Publication
(10) Pub. No.: US 2026/0305602 A1
(43) Pub. Date: Oct. 8, 2026

(54) DEVICES AND METHODS FOR CONTROLLED RELEASE OF VOLATILE ACTIVE INGREDIENTS

(71) Applicant: Noel ELMAN, Brookline, MA (US)

(72) Inventor: Noel ELMAN, Brookline, MA (US)

(21) Appl. No.: 19/484,889

(22) PCT Filed: May 17, 2024
(86) PCT No.: PCT/IB2024/054794

§ 371 Date: Nov. 17, 2025

Related U.S. Application Data
(60) Provisional application No. 63/502,928, filed on May 18, 2023.

(51) Int. Cl. A01M 99/00; A61L 9/03; A61L 9/04; A61L 9/12

(52) U.S. Cl. CPC A01M 99/00; A61L 9/03; A61L 9/035; A61L 9/037; A61L 9/048; A61L 9/12; A61L 9/122; A61L 9/125; A61L 9/127; A61L 2209/132; A61L 2209/133; A61L 2209/15

(57) ABSTRACT

Controlled release devices (CRDs) for release of a volatile active ingredient (AI) or a formulation thereof, the CRD comprising a matrix that includes the AI or the formulation thereof, and a cartridge that encases the matrix, the cartridge having pores dimensioned to control a rate of release of vapor of the volatile AI or the formulation thereof and to prevent leakage of liquid from the AI or the formulation thereof, the cartridge further having an external hydrophobic surface to prevent water penetration into the CRD.

### CROSS REFERENCE TO RELATED APPLICATIONS

This application is a 371 application from international patent application PCT/IB2024/054794 filed May 17, 2024, and is related to and claims the benefit of priority from U.S. Provisional patent application 63/502,928 filed May 18, 2023, which is incorporated herein by reference in its entirety.

### FIELD

Embodiments disclosed herein relate to devices and methods for controlled release of volatile active ingredients (AI) while preventing liquid leakage and environment water incorporation.

### BACKGROUND

The problem of delivering AIs in a controlled release manner is known and has been addressed in the past in various ways such as controlled release devices (CRD) for vector control in agricultural, military, or civilian applications.

An example showing efficacy of CRDs is given in Stevenson, Jennifer C., et al. “Controlled release spatial repellent devices (CRDs) as novel tools against malaria transmission: a semi-field study in Macha, Zambia.” Malaria Journal 17.1(2018 ): 437. Another example of CRD implementation is given in Bernier, Ulrich, et al. “Combined Experimental-Computational Approach for Spatial Protection Efficacy Assessment of Controlled Release Devices against Mosquitoes (Anopheles),” PLoS Negl Trop Dis. 2019 Mar. 11;13(3).

The challenges facing development of effective CRDs include controlling the release rate of the AI from within the CRD, and preventing activation of the AI within the CRD until the CRD is activated (used). The AI may be loaded into a CRD as a mixture together with other possible materials (“AI formulation”), to achieve optimal properties for a given application. Furthermore, since many AI are volatile organic compounds, there is a need to contain the AI in a liquid form within the CRD, while allowing its release only in a gaseous phase and while preventing leakage and external water incorporation.

A long shelf life and an efficient activation deactivation mechanism are required to maximize duration and protection.

Further, there is a need for CRDs that are inexpensive, environmentally friendly, and easy to manufacture and assemble.

### SUMMARY

Exemplary embodiments (or “examples”) disclosed herein relate to CRDs, a system (array of CRDs) and methods for controlled release of a volatile active ingredient (AI) or formulations thereof by active or passive mechanisms. The release of the AI or formulation thereof is from within the CRD into a fluid environment. In some examples, the CRD is wearable. Exemplarily, the fluid environment is air. Henceforth, and any description of AI also refers to the formulation thereof.

In examples, the AIs serve for any one of spatial repellents, insecticides, herbicides, larvicides, or a combination of these. Optionally, the systems, devices and methods of the present disclosure serve for the release of AIs with other functions and general applications.

Some examples provide for CRDs with multiple mechanisms for storing the formulation, controlling the release rate of an AI from within the CRD and also mechanisms for preventing activation of the AI, or a combination of the AI with other components within the CRD until the CRD is activated. When activated, a CRD is in an “active” state. When not activated, a CRD is in an “inactive” state.

In some examples, devices disclosed herein can be implemented as wearable devices for protection against vectors such as mosquitoes and ticks. In some examples, the devices can be deployed both indoors or outdoors, for applications such as: agricultural applications, for example to protect against multiple vectors that affect crops, such as weevils, or psyllids by attachment to a tree or deployment in soil; weed eradication such as use of herbicides provided in low dosage, low toxicity deliveries; floating devices to disperse larvicides to remove larvae from water, and so forth. In some examples, a device disclosed herein is manufactured from biodegradable, environmentally friendly materials.

In various exemplary embodiments there are provided CRDs for release of a volatile AI or a formulation thereof, a CRD comprising: a matrix that includes the AI or the formulation thereof; and a cartridge that encases the matrix, the cartridge having pores dimensioned to control a rate of release of vapor of the volatile AI or the formulation thereof and to prevent leakage of liquid from the AI or the formulation thereof, the cartridge further having an external hydrophobic surface to prevent water penetration into the CRD.

In some examples, a CRD further comprises a removable cover covering the cartridge and adapted to prevent the release of the volatile AI when the CRD is in an inactive state and to allow the release of the AI when the CRD is in an activate state.

In some examples, the matrix is a liquid absorbing material or structure. The liquid absorbing material or structure may be a wick, a porous sponge material, a fiber material, or a non-woven fabric. In some examples, the matrix may be a gel. In some examples, the matrix may be a cavity, i.e. an empty container.

In some examples, the pores may have a pore dimension in the range of 0.01-250 μm.

In some examples, the hydrophobic surface includes an outer coating of the cartridge made of a polymer material. The polymer material may be for example polyethylene, an acrylic polymer, a fluorocarbon polymer, a polystyrene polymer, a poly(vinyl chloride) polymer, a poly(N-vinylpyrrolidone) polymer, a polyamide, a polycarbonate, a polyester, or a polyether.

In some examples, the hydrophobic surface may be a hydrophobic material forming the cartridge. In some example, the hydrophobic material forming the cartridge may be Acrylonitrile butadiene styrene (ABS), polycarbonate, polyethylene (PE), high-density polyethylene (HDPE), polyamide-imides (PAI), high impact polystyrene (HIPS), polypropylene, polyoxymethylene (POM), or Polyether ether ketone (PEEK).

In some examples, the volatile AI may be selected from the group consisting of repellents, insecticides, larvicides, oils, natural oils, essences, and fragrances.

In some examples, the AI formulation may be selected from the group consisting of a volatile AI combined with an organic solvent (OS) or an aqueous solution, or a combination thereof.

In some examples, the AI formulation may selected from the group consisting of a volatile AI combined with a gelling agent.

In some examples, a CRD as above or below may be mechanically coupled to at least one other CRD having a respective different volatile AI or formulation thereof, forming a system operative to release of at least two different AIs or formulations thereof, either independently or concurrently, when the CRDs are in respective active states.

In some examples, the rate of release of vapor of the volatile AI or the formulation thereof out of the cartridge may be enhanced through the employment of a fan, a chemical heater, an electric heater, or an ultrasonic actuator, or a combination thereof.

In some examples, the CRD may be integrated with clothing. In some examples, the CRD may be attached to the clothing via a Velcro like attachment.

### BRIEF DESCRIPTION OF THE DRAWINGS

Aspects, embodiments and features disclosed herein will become apparent from the following detailed description when considered in conjunction with the accompanying drawings. Like elements may be numbered with like numerals in different drawings. In the drawings:

FIGS. 1A, 1B and 1C show exemplary embodiments of a controlled release device (CRD) with different geometries;

FIGS. 1D, 1E and 1F show sectional illustrations of exemplary cartridges which may be used with the CRDs of FIGS. 1A-1C, in which the AI or a formulation thereof can be stored using methods disclosed herein;

FIGS. 1G and 1H show a sectional illustration of a cartridge with a reservoir which may be used with the CRD of FIG. 1B or 1C, in which the cartridge comprises a reservoir with several matrices that can conduct the AI or a formulation thereof;

FIGS. 1I and 1J show examples of the CRD of FIG. 1A in, respectively, inactive and active states;

FIGS. 1K and 1L show examples of the CRD of FIG. 1B in, respectively, inactive and active states;

FIGS. 1M and 1N show examples of the CRD of FIG. 1C in, respectively, inactive and active states;

FIG. 2A-2C shows exemplary experimental data detailing the release rate of the AI from a cartridge of a CRD, for different cartridges, pore sizes and matrix densities;

FIG. 3A shows an exemplary embodiment of a CRD which can be integrated as a flexible encapsulated patch;

FIG. 3B shows an enlargement of the CRD of FIG. 3A;

FIG. 3C shows an enlargement of the CRD of FIG. 3A in its inactive state;

FIG. 4A shows an exemplary embodiment of a flexible CRD;

FIG. 4B shows an exemplary embodiment of the CRD of FIG. 4A integrated into clothing;

FIG. 5 shows examples of a system of CRDs attached to one another using flexible joints;

FIGS. 6A-6B show an example of a CRD wherein the AI mass transfer may be enhanced by convection;

FIGS. 7A-7D show an example of a CRD wherein the AI mass transfer may be enhanced by applying heat through an exothermic reaction;

FIGS. 8A-8D show an example of a CRD wherein the AI mass transfer may be enhanced by applying heat through an electric heater;

FIGS. 9A-9B show an example of a CRD wherein the AI mass transfer may be enhanced by applying ultrasound.

### DETAILED DESCRIPTION

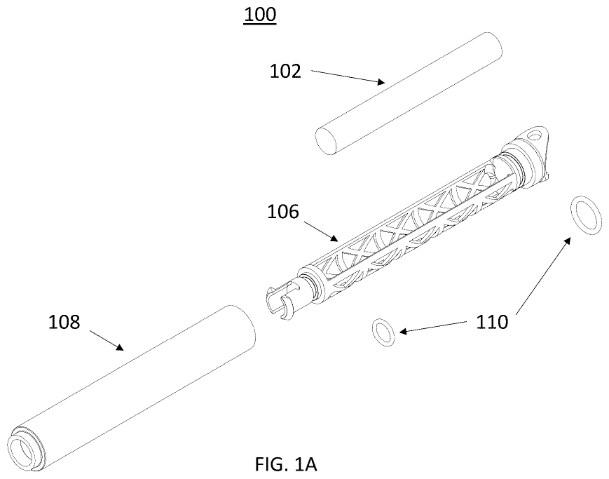

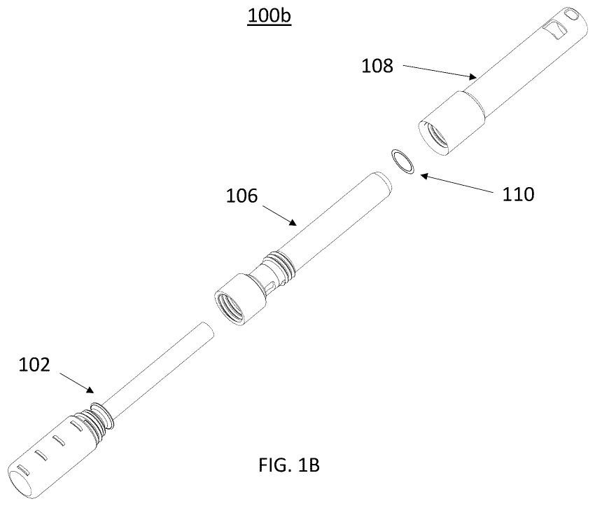

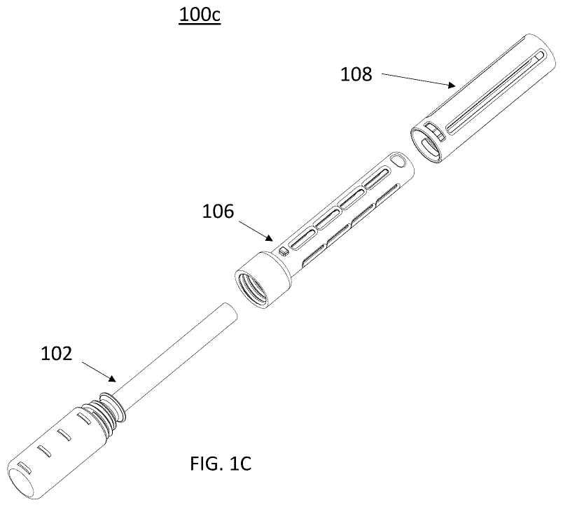

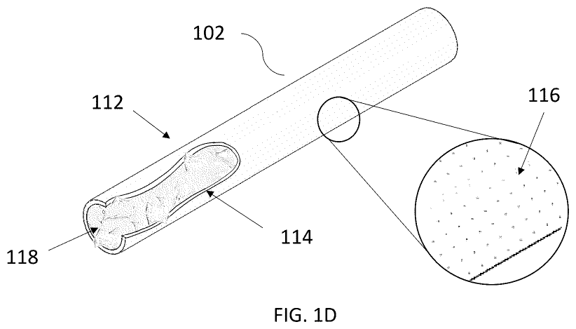

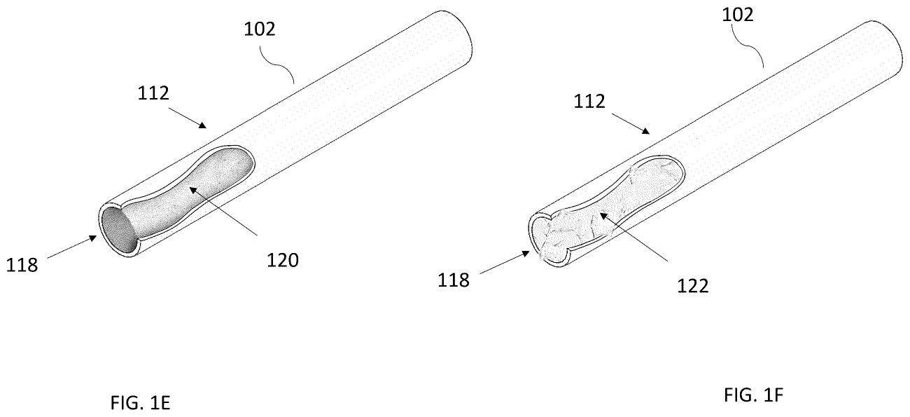

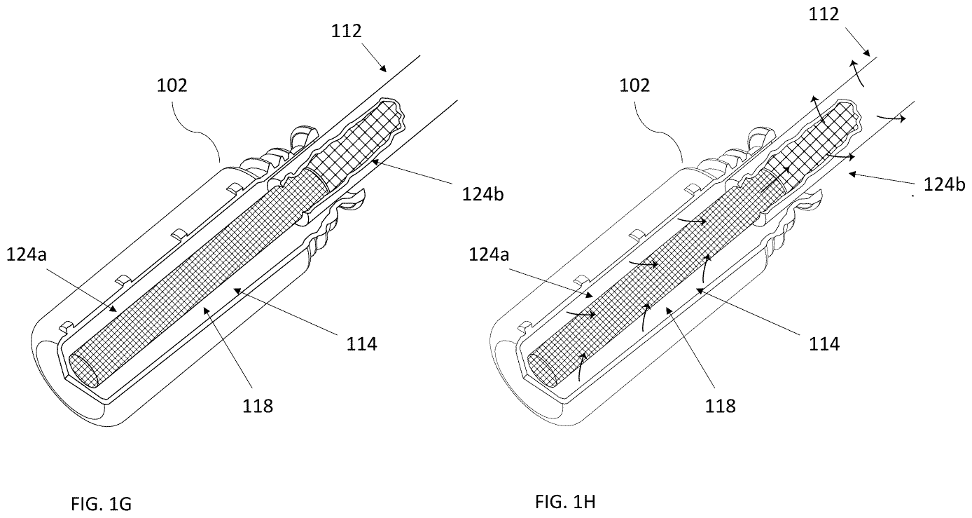

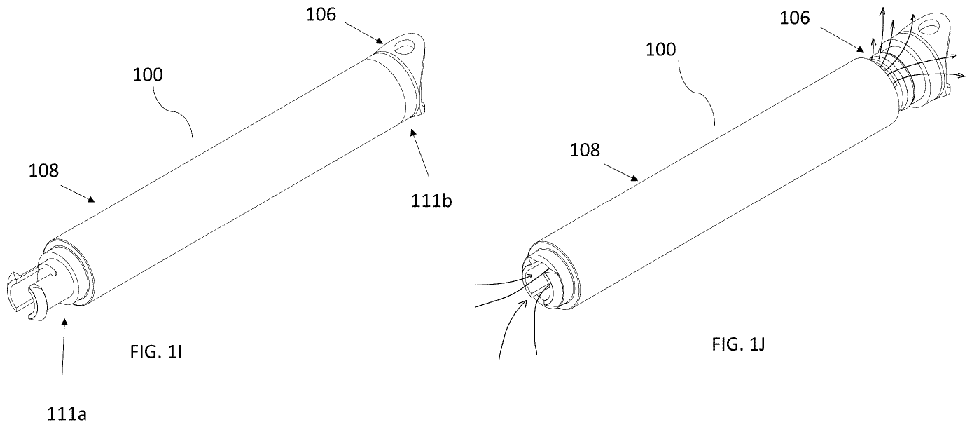

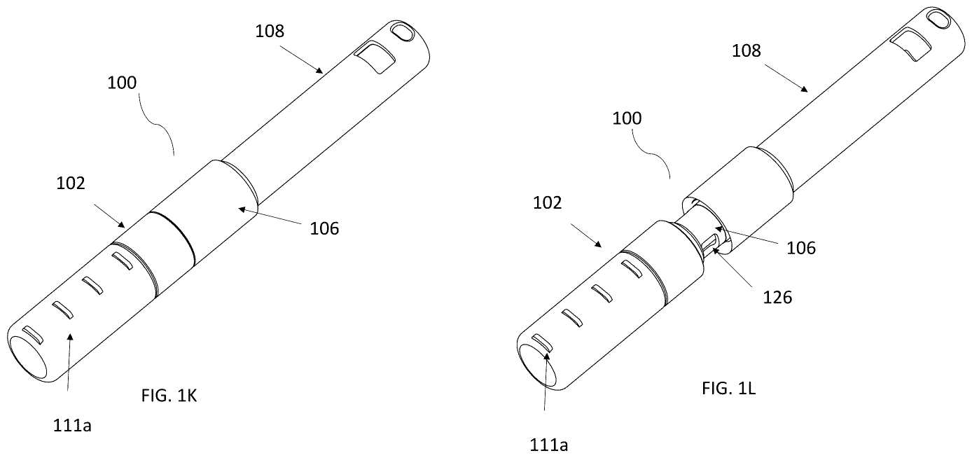

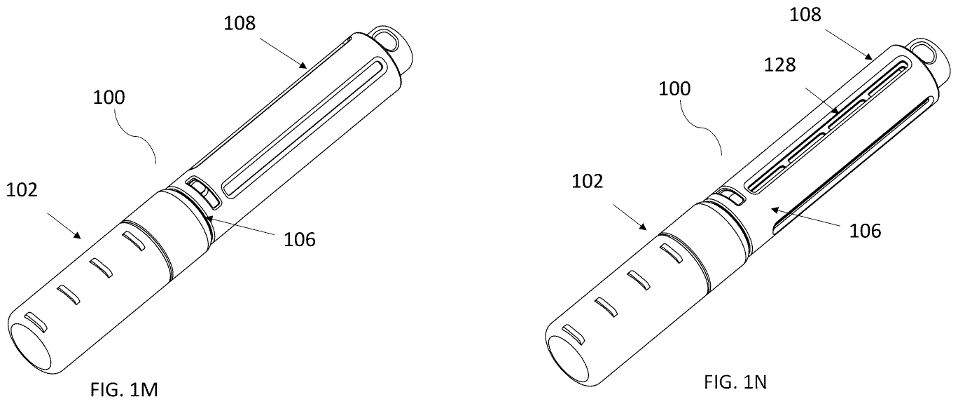

FIGS. 1A-1C show exemplary embodiments of a CRD disclosed herein, with different geometries, and numbered 100a-100c respectively. As shown, CRD 100 comprises a cartridge 102 which houses (or “encases”) a matrix that includes an AI or a formulation thereof (118, see below), the AI formulation being in some examples a mixture of the AI together with other materials. In some examples, the matrix may be a reservoir, i.e. a cavity.

Example cartridges are shown in more detail in FIGS. 1D-1F. Exemplarily, cartridge 102 has a cylindrical geometry, but other geometries are also possible. Cartridge 102 is housed in device body 106, which is covered by a cover 108, and is replaceable (i.e. disposable). Note that in some examples, the device body and the cartridge may be the same component, and all physical attributes of the device body may be implemented in the cartridge. Cover 108 prevents the release of the AI formulation by keeping the CRD in an inactive state, by preventing AI contact with the fluid environment, as shown in FIGS. 1I, 1K and 1M. The prevention of the AI release in the inactive CRD state may be achieved by hermetically sealing the CRD, for example with seals 110 at two CRD ends 18A and 18B. In the example of FIG. 1A, the CRD is hermetically sealed with seals in the form of O-rings 110. In other embodiments, seals 110 may have other geometries. In yet other embodiments, the CRD may be hermetically sealed using various methods, such as, but not limited to, ultrasonic bonding of caps. Exemplarily, cartridge 102, device body 106 and cover 108 form a concentric structure, but other geometries are also possible.

Examples of cartridges are shown in more detail in FIGS. 1D-1F. In these figures, cartridge 102 has an external permeable membrane 112 which surrounds or “covers” a reservoir 114. In some examples, reservoir 114 may include or be a matrix that houses the AI formulation, or an empty space into which the AI formulation is loaded. Membrane 112 comprises openings 116 that are sized so as to control the release rate of the AI or formulation thereof. Openings 116 may be pores, slits, or other shaped openings, and of a size such that they are visible, or may be “microscopic” (i.e. smaller than visible to a naked eye). The openings may also have different geometries. Exemplarily, openings 208 may be in the range of 0.1-100 μm. The AI diffuses out of the CRD through opening 116 when CRD 100 is placed in an active state. Openings 116 may have different geometries, sizes, orientations and directionality, in accordance with a desired release rate.

Examples of an AI include, but are not limited to, insecticides or insect repellant or acaricide (such as, but not limited to transfluthrin, metofluthrin or permethrin). Such an AI may be combined with organic solvents, such as, but not limited to, isopropyl alcohol (IPA), as well as with other materials, to create a gel formulation of AI, as described in more detail below.

Membrane 112 serves as a diffusion barrier for the AI formulation and comprises at least one hydrophobic layer. The hydrophobic layer may be achieved for example, but not limited to, by coating the outside of membrane 112 with a hydrophobic material, such as, but not limited to, hydrophobic silicon dioxide and silica nano-coatings, Manganese oxide polystyrene (MnO2/PS) nano-composites, Zinc oxide polystyrene (ZnO/PS) nano-composites, Precipitated calcium carbonate, Carbon nano-tube structures, Fluorinated silanes and Fluoropolymer coatings. Alternatively, in some examples, membrane 112 may be made out of a hydrophobic material, such as, but not limited to, polyethylene, or other polymer materials such as Acrylic Polymers, Fluorocarbon Polymers, Polystyrene Polymers, Poly(vinyl chloride) Polymers, Poly(N-vinylpyrrolidone) (PVP) Polymers, Polyamides, Polyimides, Polycarbonates, Polyesters or Polyethers. In some examples, membrane 112 can be formulated out of or coated with a hydrophobic material, as described above, in combination with additional materials such as, but not limited to, metals, carbon fibers, and silicon-based materials, such as silicon nitride. Additionally, in some examples, membrane 112 may include multiple hydrophobic layers. The hydrophobic layer, e.g. polyethylene, SiO₂, prevents water penetration into the CRD while the CRD is in its active state. Alternatively, in other examples, the hydrophobic layer may be achieved by formulating cartridge 102 out of a hydrophobic material, such as, but not limited to, a high impact polymer. Exemplary polymers may include Acrylonitrile butadiene styrene (ABS), polycarbonate, high-density polyethylene (HDPE), Polyamide-imides (PAI), high impact Polystyrene (HIPS), polypropylene, and Polyoxymethylene (POM), and ultra-high molecular weight polyethylene.

FIGS. 1E-1F show other examples of cartridge 102, which may be used with CRD 100. In these cartridges, reservoir 114 is filled with an AI formulation 118. In some examples, AI formulation 118 may be absorbed in a matrix 120, as described in FIG. 1E. Matrix 120 may comprise an absorbent medium such as a wick or porous sponge that includes AI formulation 118, for example but not limited to cellulose. In a non-limiting example, matrix 120 comprises a wick, wherein the matrix has a density that is typically provided in a range of 0.1 g/cm³ to 1 g/cm³. Matrix 120 may hold AI formulation 118 by absorption-adsorption mechanisms. Matrix 120 may be optionally provided with a high surface to volume ratio for increasing the surface area for evaporation of AI formulation 118. Alternatively, matrix 120 may comprise a synthetic material such as but not limited to Polyurethane (ether & ester grades), Microcellular urethanes, reticulated polyurethane foam filters, Crosslink polyethylene roll stock, Crosslink polyethylene, and/or Polyurethane.

In other examples, AI formulation 118 may be inserted directly into reservoir 114 in a gel form 130, as described in FIG. 1F, in which the AI is mixed with a gelling agent together with other possible materials. Formulation of the AI in gel form increases the formulation viscosity, which therefore increases the surface tension. The resultant surface tension of the AI formulation is sufficient to prevent leakage in a non-gaseous form for a given size and geometry of openings 116.

In cases that the capillary force acting on AI formulation 118 are not sufficient to prevent liquid leakage for a given pore size, to prevent the leakage, the formulation may be absorbed in a matrix 120, while still allowing controlled release of the AI through volatilization. In examples (as mentioned above) in which the AI formulation 118 is formulated as a gel 122, this is done in order to increase the overall viscosity (and thus increase the surface tension) and prevent liquid leakage, while still allowing controlled release of the AI through volatilization. Other examples may include absorption of a gel form 122 of AI formulation 118 in matrix 120. Additionally, the surface tension of AI formulation 118 can further be modulated by adding a surfactant to the formulation.

FIG. 1G shows an additional example of cartridge 102, which may be used with the CRD of FIG. 1B or 1C. Said cartridge comprises a reservoir 114 filled with an AI formulation 118. Exemplarily, AI formulation 118 is a liquid or a gel. AI formulation 118 may be conducted through a series of matrices 124a and 124b, from the reservoir 114 to permeable membrane 112. Matrices 124 are similar to matrix 120. Exemplarily, cartridge 102 comprises two matrices, but additional matrices are also possible. The fluid flow direction (signified by arrows) is described in FIG. 1H. In a non-limiting example, matrix materials 124 comprises a wick, wherein the matrix has a density that is typically provided in a range of 0.1 g/cm³ to 1 g/cm³. Matrix materials 124 conduct AI formulation 118 by capillary mechanisms. Matrix materials 124 may be optionally provided with a high surface to volume ratio for increasing the surface area for evaporation of AI formulation 118 through membrane 112. In some examples, AI formulation 118 may be fully absorbed in matrix materials 124, leaving reservoir 114 filled with gas, which exemplarily, is air. Alternatively, matrix materials 124 may comprise a synthetic material such as but not limited to Polyurethane (ether & ester grades), Microcellular urethanes, reticulated polyurethane foam filters, Crosslink polyethylene roll stock, Crosslink polyethylene, and/or Polyurethane. Matrices 124 may also be identical to matrix 120 described above. In other examples, the final matrix 124 in the series may be covered by a permeable membrane 112. In additional examples, the AI may be formulated as a gel 122 instead of or in addition to one or all of the matrices 124.

In some examples, AI formulation 118 may comprise an insecticide or insect repellant, such as transfluthrin or metofluthrin, combined with an organic solvent, such as, isopropyl alcohol (IPA). Exemplarily, as transfluthrin has a relatively high melting point of 32 degrees Celsius, it is formulated with a volatile organic solvent, which exemplary is IPA. In a non-limiting example, an AI formulation 118 of transfluthrin or metofluthrin with a volatile organic solvent, the AI is provided in a range of 1%-99% (w/w) of the formulation and the solvent in a corresponding range of 99% to 1% (w/w). The AI formulation may comprise additional pyrethroids such as, but not limited to, Allethrin, Bifenthrin, Cyhalothrin, Lambda-cyhalothrin, Cypermethrin, Cyfluthrin, Deltamethrin, Etofenprox, Fenvalerate, Permethrin, Phenothrin, Prallethrin, Resmethrin, Tetramethrin, or Tralomethrin. Other non pyrethroid agents may include Flavesone, Nootkatone, VUAA-excito-repellents, etc.

In other examples, in which AI formulation 118 is provided in gel form 122, comprising an AI as above or below. In a non-limiting example, the AI (e.g. transfluthrin) may be formulated as a gel 122 by dissolving in an organic solvent such as IPA, at a concentration of 30% (w/w). The solution may be mixed slowly with a gel solution, comprised of a gelling agent, such as, but not limited to, Carbopol, dissolved in water together with triethylamine (TEA). The AI formulation may formulated using additional pyrethroids, as listed above. In a non-limiting example of a gel AI formulation 122, where the AI is transfluthrin, the transfluthrin is provided in a range of 5%-30% (w/w).

FIGS. 1I and 1J show examples of the CRD 100 of FIG. 1A in inactive and active states, respectively. In the inactive state, cover 108 covers cartridge 102. The CRD is hermetically sealed by seals 110 at ends 18A and 18B. Cover 108 covers pores 116 of permeable membrane 112, and thus prevents the volatile release of AI formulation 118, which is housed in reservoir 114. The CRD may be placed in an active state, as shown in FIG. 1F, which results in removal of cover 108 and exposure of cartridge 102 to a fluid environment such as air. This allows the release of AI formulation 118 through passive volatilization and outwards diffusion into the fluid environment through openings 116. As a non-limiting example, upon placing CRD 100 in an active state, cover 108 is removed from device body 106 which houses cartridge 102. Exemplarily, cartridge 102 houses an AI formulation containing transfluthrin, which is absorbed in matrix 120 in the form of a cellulose wick, and is exposed to a fluid environment such as air in this active state. AI formulation 118 volatizes and the AI diffuses through openings 116, which may for example be microscopic pores of permeable membrane 112, into the air.

FIGS. 1K and 1L show examples of the CRD 100 of FIG. 1B in inactive and active states, respectively. In a similar manner to the one described above, activation of the CRD results in exposure of the AI formulation 118 to the fluid environment, resulting in passive volatilization. In this example, CRD 100 is activated by unscrewing cover 108 device body 106, which encloses cartridge 102. In this embodiment, device body 106 comprises an opening 126, through which AI formulation may diffuse once the device is activated. In a non-limiting example, AI formulation 118 in conducted through a series of matrices 124 and diffuses through opening 126 upon volatilization. In other examples, the final matrix 124 in the series may be covered by a permeable membrane 112. In additional examples, the AI may be formulated as a gel 122 instead of or in addition to one or all of the matrices 124. Exemplarily, cartridge 102 houses an AI formulation containing transfluthrin in liquid form, which is conducted through a series of matrix materials 124 to the permeable membrane 112. AI formulation 118 volatizes and the AI diffuses through openings 116, which comprise microscopic pores of permeable membrane 112 into the air. FIGS. 1M and 1N show examples of the CRD 100 of FIG. 1C in inactive and active states, respectively. The device is activated in a similar manner to the one described above, expect in this embodiment the device is activated by twisting cover 108, hereby exposing slits 128 through which the AI formulation may diffuse once volatized.

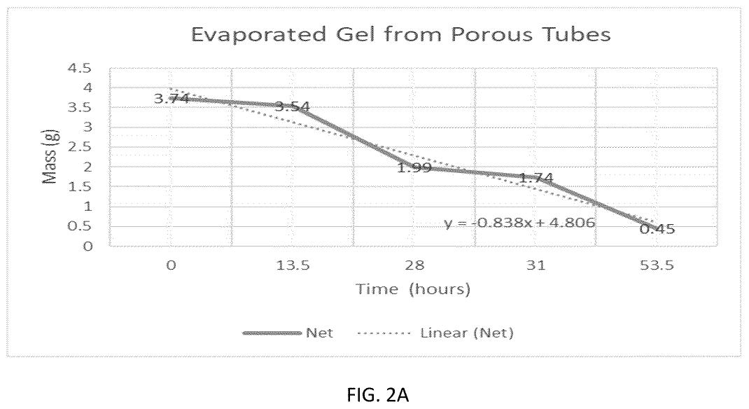

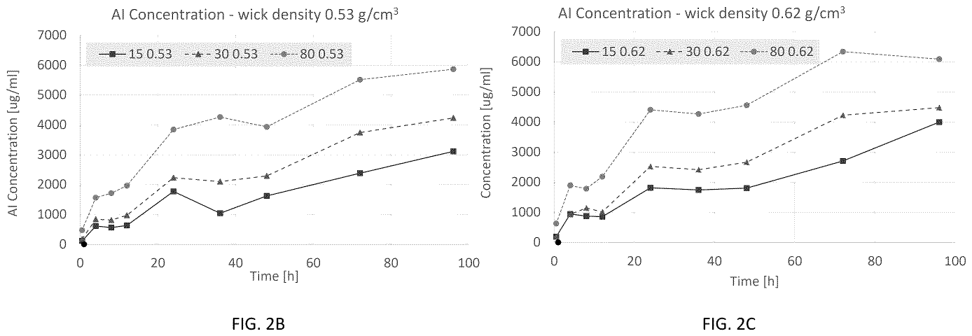

FIG. 2A shows exemplary experimental data detailing the release rate of an AI formulation 200 from a cartridge 202, which can be loaded into a CRD, and is similar to cartridge 102 described above. Exemplarily, cartridge 202 has a cylindrical geometry, and is a porous tube which comprises release pores of microscopic order which exemplarily were 80 microns in size. Exemplarily, AI formulation 200 is a gel formulation of transfluthrin, formulated with an organic solvent IPA, and with a gelling agent Carbopol, which results in a gel-based AI formulation, similarly described above. The experimental data details the release rate of the AI, expressed as the mass of the AI formulation 200, as a function of time. Exemplarily, formulation 200 was exposed to air at room temperature, and allowed to diffuse through release pores 404,

FIGS. 2B and 2C shows a graph of exemplary release rates for an AI as a function of time for different cartridges, comprising porous membranes which cover an internal reservoir, with varying pore sizes, through which the AI can diffuse. Three cartridges were tested, with pore sizes of 15, 30 and 80 microns. The AI was Transfluthrin formulated with IPA at a concentration of 30% (w/w). The AI formulation was housed in a matrix, comprised of a cellulose wick. The formulation was housed in wicks with varying densities. Exemplarily, the wick densities used were 0.53 g/cm³, shown in FIG. 2B, and 0.62 g/cm³, shown in FIG. 2CB, for a total of 6 combinations. Measurements were performed via gas chromatography-mass spectrometry, in order to detect the increasing AI concentration as a function of time. The curves indicate that the release rate of the AI can be controlled by modifying both the matrix density and the pore size, both of which affect the release kinetics. In general, it can be seen that both wick density and pore size are positively correlated with AI concentration. Experiments were conducted at room temperature.

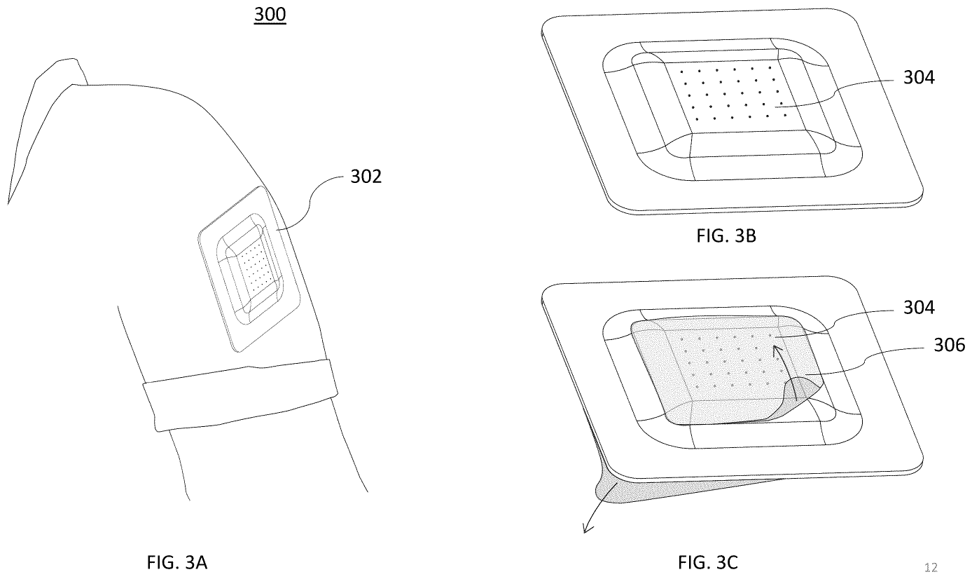

FIG. 3A shows examples of a CRD 300 disclosed herein. CRD 300 is similar to CRD 100 described above, and comprises a device body 302 which houses an AI or a formulation thereof in one of the methods described above. CRD 300 can be integrated as a flexible encapsulated patch, that may be adapted for different uses, such as, but not limited to, integrated into clothing. Exemplarily, CRD is integrated through the use of a flexible substrate which allows it to conform to clothing as a wearable device. CRD 300 may be integrated into clothing through different methods, such as, but not limited to, the use of Velcro-like attachments, or by physically sewing the device into the fabric, or by using an adhesive.

FIG. 3B shows an enlargement of device body 302, which comprises openings 304 that are sized so as to control the release rate of the AI or formulation thereof. Openings 304 may be visible, such as, but not limited to slits, but are optionally microscopic or nanoscopic permeable paths, such as release pores, through which the AI may diffuse outwards when the CRD of FIG. 3A is placed in an active state. Openings 304 may also comprise different geometries, sizes, orientations and directionality, in accordance with the desired release rate. Exemplarily, openings 304 comprise release pores that are of microscopic order, and are in the range of 0.1-100 μmin diameter. Alternatively, the release pores may also be visible openings.

FIG. 3C shows an enlargement of device body 302 in its inactive state. In the inactive state, openings 304 are covered by seal 306, and thus prevents the volatile release of the AI formulation which is housed in device body 302. The CRD may be placed in an active state by breaking or removing seal 306, which results in exposure of the device to the fluid environment. This allows the release of the AI formulation through volatilization and outwards diffusion into the fluid environment through openings 304. Exemplarily, seal 306 may be removed manually, such as, but not limited to, the use of a pull-tab.

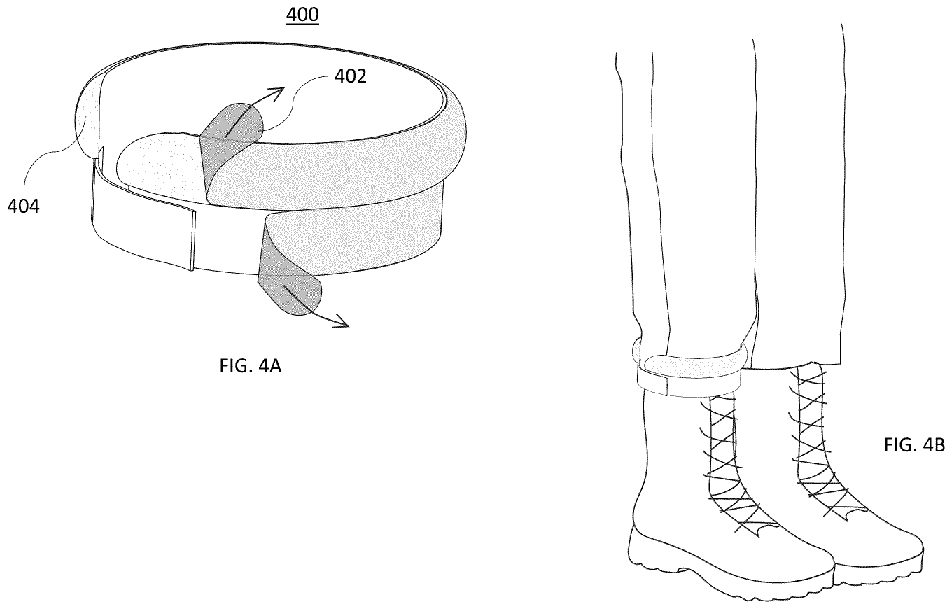

FIG. 4A shows an exemplary embodiment of a flexible CRD 400, which is similar to CRD 100 and 300, and can be placed in an active state by removal of patch 402. The AI formulation is housed in device body 404, which comprises a porous structure. By removal of patch 402, the AI formulation is released through volatilization and outwards diffusion into the fluid environment.

FIG. 4B shows an exemplary embodiment of the conformal CRD of FIG. 4A integrated into clothing. In a non-limiting example, the CRD is attached to the clothing via a Velcro-like attachment.

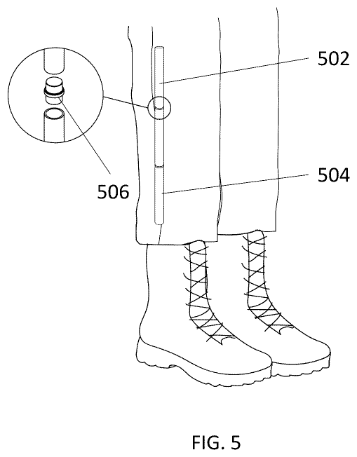

FIG. 5 shows examples of an array of CRDs 502 and 504 disclosed herein, which are similar to CRD 100 described above, that can be attached to one another using flexible joineries 506. This allows multiple CRDs to be mechanically connected to each other in series, for versatile deployment of multiple AI formulations. In a non-limiting example, CRD 502 deploys DEET or permethrin, which are contact repellents, while CRD 504 deploys transfluthrin or metofluthrin, which are spatial repellents. This option for connectivity allows this system to be deployed as a flexible wearable device, which is particularly relevant when attaching it to a flexible platform that requires conformality and flexibility, e.g., clothing.

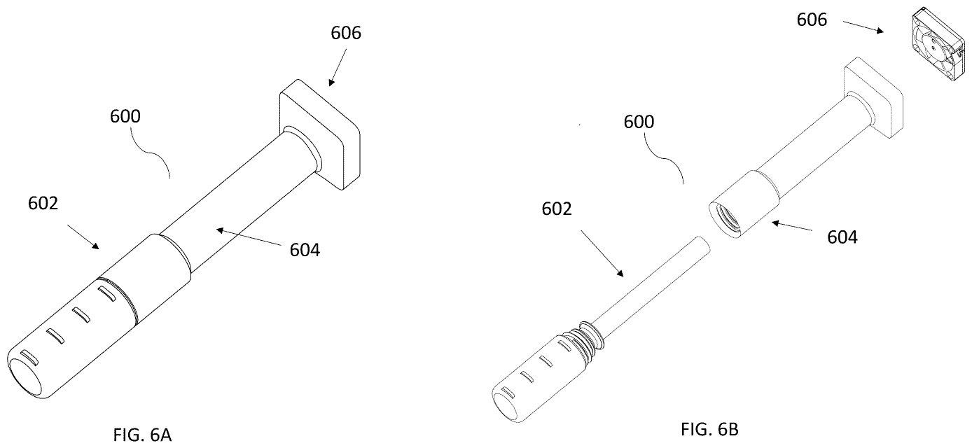

FIGS. 6A-6B show an example of a CRD 600, similar to CRD 100 described above, wherein the AI volatilization is not only passive but may be enhanced by convection. In this example, cartridge 602, which is similar to cartridge 102, is housed in device body 604. At the base or top of device body 604 is a fan 606, which may be turned on by the device user. Fan 606 may be battery powered or alternatively rechargeable. Once turned on, fan 606 blows air across the device 600, thereby enhancing convection (by air supply or extraction) and enhancing AI mass transportation. It should be noted that CRD 600 may also be used passively, similar to CRD 100, and may be activated for passive volatilization as described in FIGS. 1I-1N. Cartridge 602 may house the AI formulation in any of the configurations described above, specifically in FIGS. 1D-1H. The AI may be formulated in any of the manners described above, and may be stored in liquid form, or absorbed in a matrix, or formulated as a gel, or a combination thereof.

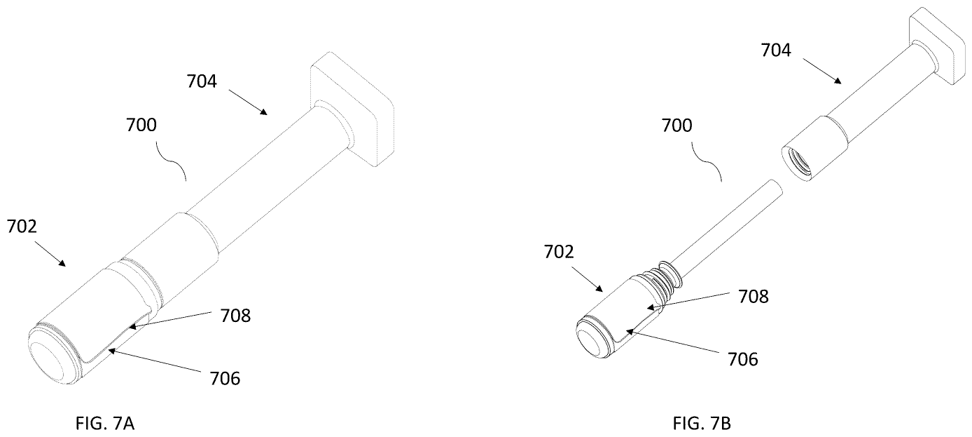

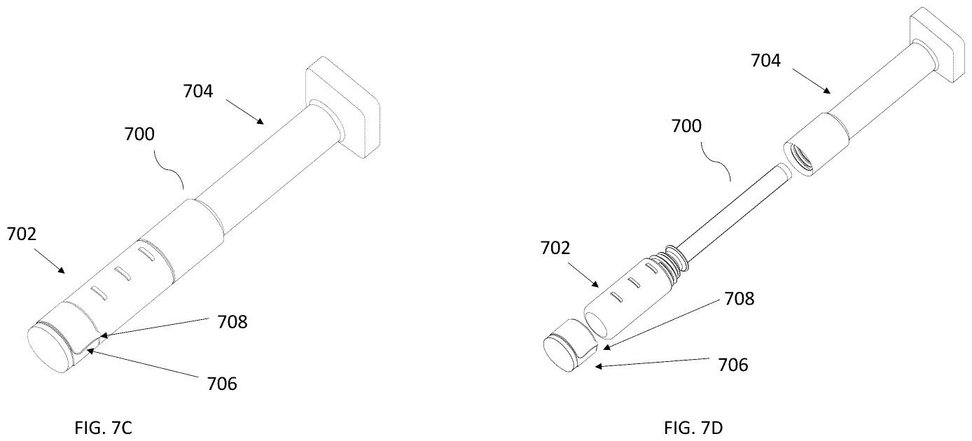

FIGS. 7A-7D show an example of a CRD 700, similar to CRD 100 and 600 described above, wherein the AI volatilization is not only passive but may be enhanced by applying heat. In this example, cartridge 702, which is similar to cartridge 102, is housed in device body 704. At the base of cartridge 702 is pad 706, which is coated with or comprised of an exothermic material, such as, but not limited to, thermite, saturated sodium acetate, exothermic redox reactions, and so on. The heating pad may be activated by the device user by using activation pull tab 708, to enhance protection through increased AI volatilization. Additional geometries of CRD 700 are described in FIGS. 7C-7D. It should be noted that CRD 700 may also be used passively, similar to CRD 100, and may be activated for passive volatilization as described in FIGS. 1I-1N. To increase heat conduction in the wick or gel, heat conductive particles, such as, but not limited to, iron microparticles, may be integrated into the matrix, cartridge reservoir, or AI gel formulation, or a combination thereof. This may also be utilized in the other CRD described above, to increase thermal conduction from the fluid environment.

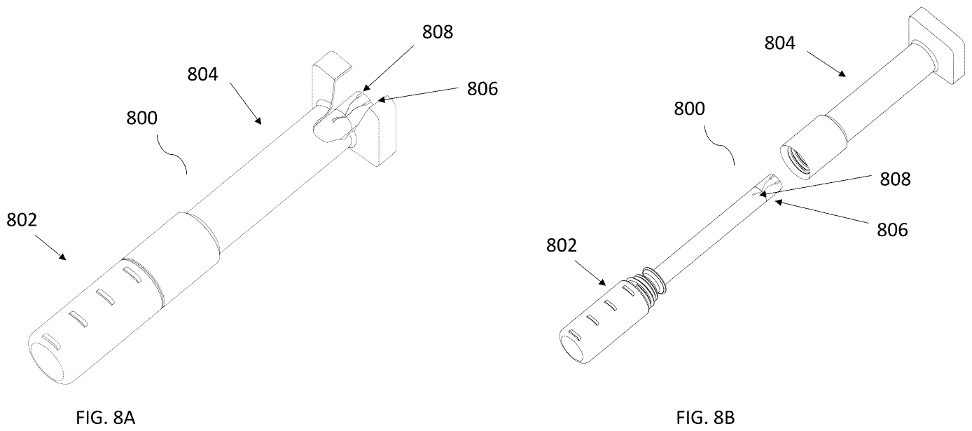

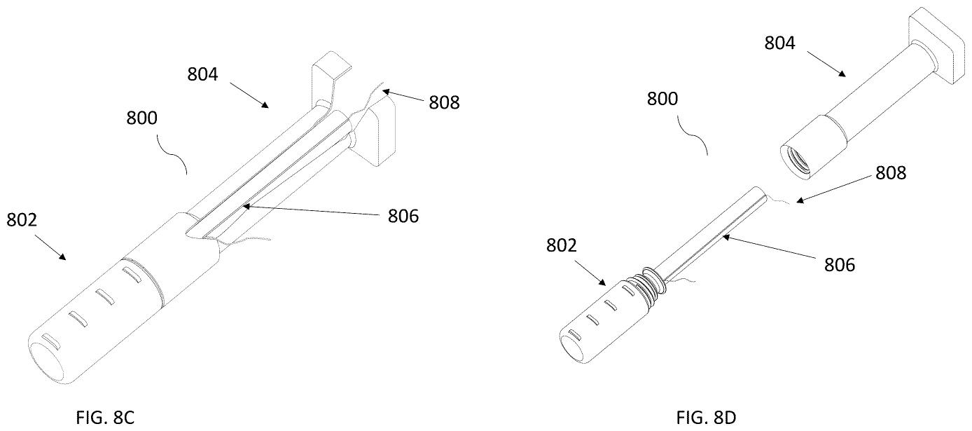

FIGS. 8A-8D show an example of a CRD 800, similar to CRD 100 and 700 described above, wherein the AI volatilization is not only passive but may be enhanced by applying heat through an electric heater. In this example, cartridge 802, which is similar to cartridge 102, is housed in device body 804. Cartridge 802 may be heated by heater 806, which is powered by DC input 808. The heater ring may be activated by the device user, to enhance protection through increased AI volatilization. Additional geometries of CRD 800 are described in FIGS. 8C-8D. It should be noted that CRD 800 may also be used passively, similar to CRD 100, and may be activated for passive volatilization as described in FIGS. 1I-1N.

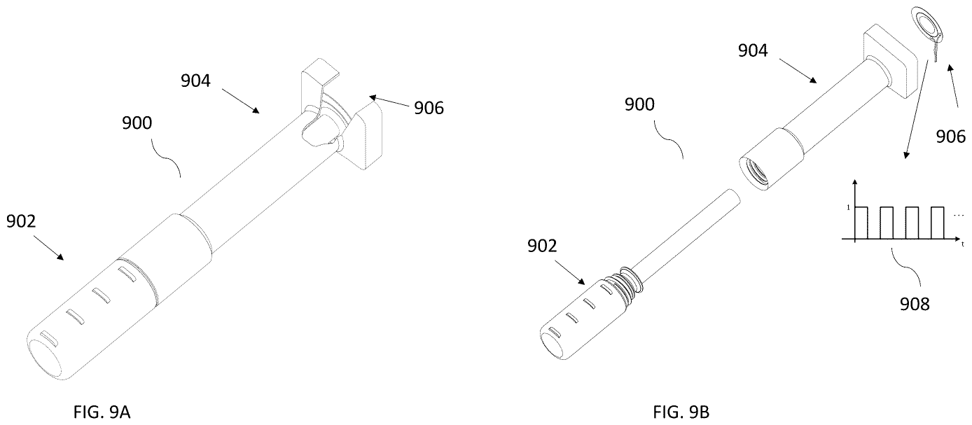

FIGS. 9A-9B show an example of a CRD 900, similar to CRD 600 and 800 described above, wherein the AI volatilization is not only passive but may be enhanced by applying ultrasound through an ultrasonic actuator. In this example, cartridge 902, which is similar to cartridge 602, is housed in device body 904. At the base or top of device 904 is ultrasonic actuator 906, which may apply an ultrasound input signal 908, to atomize the formulated AI and enhance the mass transfer of the AI into air. The actuator may be activated by the device user, to enhance protection through increased AI volatilization. It should be noted that CRD 900 may also be used passively, similar to CRD 100, and may be activated for passive volatilization as described in FIGS. 1I-1N.

In the claims or specification of the present application, unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended.

It should be understood that where the claims or specification refer to “a” or “an” element, such reference is not to be construed as there being only one of that element.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

While this disclosure describes a limited number of embodiments, it will be appreciated that many variations, modifications and other applications of such embodiments may be made. The disclosure is to be understood as not limited by the specific embodiments described herein, but only by the scope of the appended claims.

What is claimed is:

1. A controlled release device (CRD) for release of a volatile active ingredient (AI) or a formulation thereof, the CRD comprising:
a matrix that includes the AI or the formulation thereof; and
a cartridge that encases the matrix, the cartridge having pores dimensioned to control a rate of release of vapor of the volatile AI or the formulation thereof and to prevent leakage of liquid from the AI or the formulation thereof, the cartridge further having an external hydrophobic surface to prevent water penetration into the CRD.

2. The CRD of claim 1, further comprising a removable cover covering the cartridge and adapted to prevent the release of the volatile AI when the CRD is in an inactive state and to allow the release of the AI when the CRD is in an activate state.

3. The CRD of claim 1, wherein the matrix is a liquid absorbing material or structure.

4. The CRD of claim 3, wherein the liquid absorbing material or structure is selected from the group consisting of a wick, a porous sponge material, a fiber material, and a non-woven fabric.

5. The CRD of claim 1, wherein the matrix is a gel.

6. The CRD of claim 1, wherein the matrix is a cavity.

7. The CRD of claim 1, wherein the pores have a pore dimension in the range of 0.01-250 μm.

8. The CRD of claim 1, wherein the hydrophobic surface includes an outer coating of the cartridge made of a polymer material.

9. The CRD of claim 8, wherein the polymer material is selected from the group consisting of polyethylene, an acrylic polymer, a fluorocarbon polymer, a polystyrene polymer, a poly(vinyl chloride) polymer, a poly(N-vinylpyrrolidone) polymer, a polyamide, a polycarbonate, a polyester, and a polyether.

10. The CRD of claim 1, wherein the hydrophobic surface is a hydrophobic material forming the cartridge.

11. The CRD of claim 10, wherein the hydrophobic material forming the cartridge is selected from the group consisting of Acrylonitrile butadiene styrene (ABS), polycarbonate, polyethylene (PE), high-density polyethylene (HDPE), polyamide-imides (PAI), high impact polystyrene (HIPS), polypropylene, polyoxymethylene (POM), and Polyether ether ketone (PEEK).

12. The CRD of claim 1, wherein the volatile AI is selected from the group consisting of repellents, insecticides, larvicides, oils, natural oils, essences, and fragrances.

13. The CRD of claim 1, wherein the AI formulation is selected from the group consisting of a volatile AI combined with an organic solvent (OS) or an aqueous solution, or a combination thereof.

14. The CRD of claim 1, wherein the AI formulation is selected from the group consisting of a volatile AI combined with a gelling agent.

15. A system comprising the CRD of claim 1 mechanically coupled to at least one other CRD having a respective different volatile AI or formulation thereof, wherein the system is operative to release of at least two different AIs or formulations thereof, either independently or concurrently, when the CRDs are in respective active states.

16. The CRD of claim 1, wherein the rate of release of the vapor of the volatile AI or the formulation thereof is enhanced through the employment of a fan, a chemical heater, an electric heater, or an ultrasonic actuator, or a combination thereof.

17. The CRD of claim 1, wherein the CRD is integrated with clothing.

18. The CRD of claim 17, wherein the CRD is attached to the clothing via a Velcro like attachment.
